(19) United States
(12) Patent Application Publication
(10) Pub. No.: US 2004/0053265 A1
(43) Pub. Date: Mar. 18, 2004

(54) Diagnostic and therapeutic use of a caveolae-associated integral membrane protein for alzheimer's disease and related neurodegenerative disorders

(76) Inventors: Rainer Hipfel, Hamburg (DE); Heinz Von Der Kammer, Hamburg (DE); Ralf Krappa, Hamburg (DE); Johannes Pohlner, Hamburg (DE)

Correspondence Address: JACOBSON HOLMAN PLLC, 400 SEVENTH STREET N.W., SUITE 600, WASHINGTON, DC, 20004, US

(21) Appl. No.: 10/362,129

(22) PCT Filed: Aug. 24, 2001
(86) PCT No.: PCT/EP01/09802

(30) Foreign Application Priority Data
Aug. 24, 2000 (EP) 00118443.1

(51) Int. Cl. C12Q 1/68; G01N 33/53; G01N 33/567

(52) U.S. Cl. 435/6; 435/7.2

(57) ABSTRACT

The present invention discloses the differential expression of the caveolae-associated integral membrane protein flotillin-1 gene in specific brain regions of Alzheimer's disease patients. Based on this finding, this invention provides a method for diagnosing or prognosing Alzheimer's disease or other neurodegenerative diseases in a patient, or for determining whether a subject is at increased risk of developing Alzheimer's disease or other related neurodegenerative diseases. Furthermore, this invention provides therapeutic and prophylatic methods for treating or preventing Alzheimer's disease and related neurodegenerative disorders using a caveolae-associated integral membrane protein gene, in particular the flotillin-1 gene. A method of screening for modulating agents of neurodegenerative, diseases is also disclosed.

The present invention relates to methods of diagnosing, prognosing and monitoring the progression of neurodegenerative diseases in a subject. Furthermore, methods of therapy control and screening for modulating agents of neurodegenerative diseases are provided. The invention also discloses pharmaceutical compositions, kits and recombinant animal models.

Neurodegenerative diseases, in particular Alzheimer's disease, have a severely debilitating impact on a patient's life. Furthermore, these diseases constitute an enormous health, social and economic burden. Alzheimer's disease is the most common age-related neurodegenerative condition affecting about 10% of the population over 65 years of age and up to 45% over age 85 (for a recent review see Vickers et al., Progress in Neurobiology 2000, 60:139-165). Presently this amounts to an estimated 12 million cases in the US, Europe, and Japan. This situation will inevitably worsen with the demographic increase in the number of old people (“aging of the baby boomers”) in developed countries. The neuropathological hallmarks that occur in the brains of individuals with Alzheimer's disease are senile plaques, composed of amyloid-b protein, and profound cytoskeletal changes coinciding with the appearance of abnormal filamentous structures and the formation of neurofibrillary tangles. AD is a progressive disease that is associated with early deficits in memory formation and ultimately leads to the complete erosion of higher cognitive function. A characteristic feature of the pathogenesis of AD is the selective vulnerability of particular brain regions and subpopulations of nerve cells to the degenerative process.

Specifically, the temporal lobe region and the hippocampus are affected early and more severely during the progression of the disease. On the other hand, neurons within the frontal cortex, occipital cortex, and the cerebellum remain largely intact and are protected from neurodegeneration (Terry et al., Annals of Neurology 1981, 10:184-192).

Currently, there is no cure for AD, nor is there an effective treatment to halt the progression of AD or even to diagnose AD ante-mortem with high probability. Several risk factors have been identified that predispose an individual to develop AD, among them most prominently the epsilon4 allele of apolipoprotein E (ApoE). Although there are rare examples of early-onset AD which have been attributed to genetic defects in the genes for APP, presenilin-1, and presenilin-2, the prevalent form of late-onset sporadic AD is of hitherto unknown etiologic origin. The late onset and complex pathogenesis of neurodegenerative disorders pose a formidable challenge to the development of therapeutic and diagnostic agents. Therefore, it is crucial to expand the pool of potential drug targets and diagnostic markers.

It is therefore an object of the present invention to provide insight into the pathogenesis of neurological diseases and to provide methods, material and animal models which are suited inter alia for the diagnosis and development of a treatment of these diseases.

This object has been solved by the features of the independant claims. The subclaims define preferred embodiments of the present invention.

The term “and/or” used in the present specification and the claims implies that the phrases before and after this term are to be considered either as alternatives or in combination. For instance, the wording “determination of a level and/or an activity” means that either only a level, or only an activity, or both a level and an activity are determined. The term “fragment” as used herein is meant to comprise e.g. an alternatively spliced, or truncated, or otherwise cleaved transcription product or translation product. The term “derivative” as used herein is referring e.g. to a mutant, or otherwise modified transcription product and to a mutant or otherwise modified translation product, e.g. isoforms generated posttranslationally, for instance, by altered phosphorylation, or glycosylation, or lipidation, or by altered signal peptide cleavage or other types of maturation cleavage. The term “level” as used herein is meant to comprise a gage of, or a measure of the amount of, or a concentration of a transcription product, for instance an mRNA, or a translation product. The term “activity” as used herein can be understood as a measure for the ability of a transcription product or a translation product to produce a biological effect or a measure for a level of biologically active molecules. The terms “level” and/or “activity” as used herein further refer to gene expression levels or gene activity. Gene expression is defined as the complete utilization of the information contained in a gene by transcription and translation leading to production of a gene product. A gene product consists of either mRNA or protein, as a result of expression of a gene. The amount of gene product is used to measure how active a gene is.

In one aspect, the invention features a method of diagnosing or prognosing a neurodegenerative disease in a subject, or determining whether a patient is at increased risk of becoming diseased with said disease. The method comprises: determining a level, or an activity, or both said level and said activity of (i) a transcription product of a gene coding for a caveolae-associated integral membrane protein, and/or of (ii) a translation product of a gene coding for a caveolae-associated integral membrane protein, and/or of (iii) a fragment or derivative of said transcription or translation product in a sample from said subject and comparing said level, and/or said activity to a reference value The term “fragment” as used herein is meant to comprise e.g. an alternatively spliced, or truncated, or otherwise cleaved transcription product or translation product. The term “derivative” as used herein is referring e.g. to a mutant, or otherwise modified transcription product and to a mutant or otherwise modified translation product, e.g. isoforms generated posttranslationally, for instance, by altered phosphorylation, or glycosylation, or lipidation, or by altered signal peptide cleavage or other types of maturation cleavage. The term “level” as used herein is meant to comprise a gage of, or a measure of the amount of, or a concentration of a transcription product, for instance an mRNA, or a translation product. The term “activity” as used herein can be understood as a measure for the ability of a transcription product or a translation product to produce a biological effect or a measure for a level of biologically active molecules. The terms “level” and/or “activity” as used herein further refer to gene expression levels or gene activity. Gene expression is defined as the complete utilization of the information contained in a gene by transcription and translation leading to production of a gene product. A gene product consists of either mRNA or protein, as a result of expression of a gene. The amount of gene product is used to measure how active a gene is.

In one aspect, the invention features a method of diagnosing or prognosing a neurodegenerative disease in a subject, or determining whether a patient is at increased risk of becoming diseased with said disease. The method comprises: determining a level, or an activity, or both said level and said activity of (i) a transcription product of a gene coding for a caveolae-associated integral membrane protein, and/or of (ii) a translation product of a gene coding for a caveolae-associated integral membrane protein, and/or of (iii) a fragment or derivative of said transcription or translation product in a sample from said subject and comparing said level, and/or said activity to a reference value corticobasal degeneration. Another disorder featuring neurodegenerative processes is stroke.

Caveolae-associated membrane proteins are meant to comprise all proteins and polypeptides which are localized to and integrated into the membrane of caveolae, including, for instance, caveolins, flottilins, G-protein-coupled receptors, receptor tyrosine kinases, and other integral membrane signal transduction molecules. In one embodiment of the present invention, said caveolae-associated membrane proteins comprise all of the aforementioned proteins except caveolin-3. In another embodiment, said caveolae-associated membrane proteins comprise all of the aforementioned proteins except the caveolins. It is preferred that said caveolae-associated integral membrane protein is a member of the flotillin gene family, particularly flotillin-1. It is further preferred that said fragment of said caveolae-associated integral membrane protein is a fragment of flotillin-1, particularly FCRD.

The present invention discloses the novel finding of the flotillin-1 gene as being differentially expressed in the brain of Alzheimer's disease patients. Consequently, the flotillin-1 gene and its corresponding translation products may have a causative role in the regional selective neuronal degeneration. Alternatively, flotillin-1 may confer a protective function to the remaining surviving nerve cells. Based on these findings, the present invention has utility for the diagnostic evaluation and prognosis as well as for the identification of a predisposition to a neurodegenerative disease, in particular Alzheimer's disease. Furthermore, the present invention provides methods for the diagnostic monitoring of patients undergoing treatment for such a disease.

The gene for flotillin-1 was initially cloned by Bickel et al. (Journal of Biological Chemistry 1997, 272:13793-13802; the contents of which are incorporated herein by reference) in an attempt to identify genes for novel proteins that are enriched in the membranes of purified caveolae from murine lung tissue. The cDNA for murine flotillin-1 encodes a protein of 428 amino acids with a predicted molecular weight of 47 kDa. The human flotillin-1 gene sequence (Genbank Accession No. AF 089750) was deposited in the Genbank data base by A. J. Edgar (Imperial College, London, UK) by direct submission in 1998. The human flotillin-1 gene encodes a protein of 427 amino acids. The amino acid sequences of the human and murine flotillin-1 protein are highly conserved (>98%). The flotillin-1 gene displays significant homology to ESA (epidermal surface antigen, also named flotillin-2). Although flotillin-1 and ESA have different N-termini, they share a region of 47% identity on the amino acid level. Furthermore, there is a modest (˜24%) homology to two hypothetical open reading frames from the cyanobacterium Synechococcus. Flotillin-1 is expressed in most tissues and, contrary to ESA, also in brain. Bickel et al. concluded from Western blot experiments that there is evidence for a ˜27 kDa immunoreactive protein named FCRD (=flotillin cross-reacting determinant). The origin of FCRD is not known. It could either be a degradation product generated during sample preparation or a physiologically relevant proteolytic fragment of the complete flotillin-1 protein.

The flotillin-1 polypeptide is tightly associated with and enriched in caveolae membranes (i.e. an integral membrane protein). Caveolae (“little caves”) are localized invaginations or vesicular organelles with a diameter of 50-100 nm, representing a functional subcompartment of the plasma membrane (for recent reviews see Anderson, Annual Review of Biochemistry 1998, 67:199-225 and Shaul and Anderson, American Journal of Physiology 1998, 275.L843-51). Caveolae have a relatively high content of cholesterol, sphingomyelin, glycosphingolipids and lipid-anchored membrane proteins. A light bouyant density and relative resistance to solubilization by detergents such as Triton X-100 are biochemical features of caveolae. Caveolins are the principal protein components of caveolae. It has been suggested that the caveolins (caveolin-1, caveolin-2, caveolin-3; all 21-24 kDa integral membrane proteins) concentrate and organize lipids and proteins into microdomains by functioning as scaffolding units. Additionally, many signal transduction molecules such as G-protein-coupled receptors, receptor tyrosine kinases, Src-family tyrosine kinases and nitric oxide synthase have been localized to caveolae. A direct role of caveolin-1 in the regulation of GPCR-mediated signaling cascades has been shown by Carman et al. (J Biol Chem 1999, 274:8858-64) and Schreiber et al. (J Biol Chem 2000, 275:24115-23). This implies that, altogether, caveolae serve a critical function in compartmentalizing, modulating, and integrating signalling events at the cell surface. Furthermore, caveolae are implicated in the regulation of vesicular transport processes and in sorting, processing and the degradation of internalized molecules.

The 45 kDa flotillin-1 protein is a marker protein for the slightly larger caveolae-related domains (50-200 nm). Flotillins are caveolae-associated integral membrane proteins. The flotillin gene family does not exhibit any sequence homology to the caveolins. Caveolin-1 and caveolin-3 each form homo-oligomers consisting of 14-16 monomers, and when caveolins and flotillins are co-expressed within the same cell they form stable hetero-oligomeric complexes (Volonté et al., J Biol Chem 1999, 274:12702-9). Furthermore, the heterologous expression of murine flotillin-1 in insect cells is sufficient to generate caveolae-like vesicles in cell culture experiments.

It is preferred that the sample to be analyzed and determined is selected from the group comprising brain tissue or other body cells. The sample can also comprise cerebrospinal fluid or other body fluids including saliva, urine, serum plasma, or nasal mucosa.

In further preferred embodiments, said reference value is that of a level, or an activity, or both said level and said activity of (i) a transcription product of a gene coding for a caveolae-associated integral membrane protein, and/or of (ii) a translation product of a gene coding for a caveolae-associated integral membrane protein, and/or of (iii) a fragment or derivative of said transcription or translation product in a sample from a subject not suffering from said neurodegenerative disease.

In preferred embodiments, an altered amount of flotillin-1 mRNA and/or flotillin-1 protein in a sample from said subject relative to a reference value representing a known health status indicates a diagnosis, or prognosis, or increased risk of becoming diseased with a neurodegenerative disease, particularly Alzheimer's disease.

In preferred embodiments, measurement of the level of transcription products of a gene coding for a caveolae-associated integral membrane protein is performed in a sample from a subject using Northern blots with probes specific for said gene. Quantitative PCR with primer combinations to amplify said gene-specific sequences from cDNA obtained by reverse transcription of RNA extracted from a sample of a subject can also be applied. These techniques are known to those of ordinary skill in the art (see Sambrook and Russell, Molecular Cloning: A Laboratory Manual, Cold Spring Harbor Laboratory Press, Cold Spring Harbor, N.Y., 2000).

Furthermore, the level and/or activity of a translation product of said gene and/or fragment or derivative of said translation product can be detected using a Western blot, an immunoassay, an enzyme activity assay, and/or binding assay. These assays can measure the amount of binding between said protein molecule and an anti-protein antibody by the use of enzymatic, chromodynamic, radioactive, or luminescent labels which are attached to either the anti-protein antibody or a secondary antibody which binds the anti-protein antibody. In addition, other high affinity ligands may be used. Immunoassays which can be used include e.g. ELISAs, Western blots and other techniques known to those of ordinary skill in the art (see Harlow and Lane, Antibodies: A Laboratory Manual, Cold Spring Harbor Laboratory Press, Cold Spring Harbor, N.Y., 1999).

In a preferred embodiment, the level, or the activity, or both said level and said activity of (i) a transcription product of a gene coding for a caveolae-associated integral membrane protein, and/or of (ii) a translation product of a gene coding for a caveolae-associated integral membrane protein, and/or of (iii) a fragment or derivative of said transcription or translation product in a series of samples taken from said subject over a period of time is compared, in order to monitor the progression of said disease. In further preferred embodiments, said subject receives a treatment prior to one or more of said sample gatherings. In yet another preferred embodiment, said level and/or activity is determined before and after said treatment of said subject.

In another aspect, the invention features a method of treating or preventing a neurodegenerative disease, in particular Alzheimer's disease, in a subject comprising the administration to said subject in a therapeutically or prophylactically effective amount of an agent or agents which directly or indirectly affect a level, or an activity, or both said level and said activity, of (i) a gene coding for a caveolae-associated integral membrane protein, and/or (ii) a transcription product of a gene coding for a caveolae-associated integral membrane protein, and/or (iii) a translation product of said gene, and/or (iv) a fragment or derivative of (i) to (iii).

In preferred embodiments, the method comprises the application of per se known methods of gene therapy and/or antisense nucleic acid technology to administer said agent or agents. In general, gene therapy includes several approaches: molecular replacement of a mutated gene, addition of a new gene resulting in the synthesis of a therapeutic protein, and modulation of endogenous cellular gene expression by recombinant expression methods or by drugs. Gene-transfer techniques are described in detail (see e.g. Behr, Acc Chem Res 1993, 26:274-278 and Mulligan, Science, 1993, 260: 926-931; the contents of which are incorporated herein by reference) and include direct gene-transfer techniques such as mechanical microinjection of DNA into a cell as well as indirect techniques employing biological vectors (like recombinant viruses, especially retroviruses) or model liposomes, or techniques based on transfection with DNA coprecipitation with polycations, cell membrane pertubation by chemical (solvents, detergents, polymers, enzymes) or physical means (mechanic, osmotic, thermic, electric shocks). The postnatal gene transfer into the central nervous system has been described in detail (see e.g. Wolff, Curr Opin Neurobiol 1993, 3:743-748).

In particular, the invention features a method of treating or preventing a neurodegenerative disease by means of antisense nucleic acid therapy, i.e. the down-regulation of an inappropriately expressed or defective gene by the introduction of antisense nucleic acids or derivatives thereof into certain critical cells (see e.g. Gillespie, DN&P 1992, 5:389-395; Agrawal and Akhtar, Trends Biotechnol 1995, 13:197-199; Crooke, Biotechnology 1992, 10:882-6; the contents of which are incorporated herein by reference). Apart from hybridization strategies, the application of ribozymes, i.e. RNA molecules that act as enzymes, destroying RNA that carries the message of disease has also been described (see e.g. Barinaga, Science 1993, 262:1512-1514; the contents of which are incorporated herein by reference). In preferred embodiments, the subject to be treated is a human, and therapeutic antisense nucleic acids or derivatives thereof are directed against a human caveolae-associated integral membrane protein, particularly flotillin-1. It is preferred that cells of the central nervous system, preferably the brain, of a subject are treated in such a way. Cell penetration can be performed by known strategies such as coupling of antisense nucleic acids and derivatives thereof to carrier particles, or the above described techniques. Strategies for administering targeted therapeutic oligodeoxynucleotides are known to those of skill in the art (see e.g. Wickstrom, Trends Biotechnol, 1992, 10: 281-287; the contents of which are incorporated herein by reference). In some cases, delivery can be performed by mere topical application. Further approaches are directed to intracellular expression of antisense RNA. In this strategy, cells are transformed ex vivo with a recombinant gene that directs the synthesis of an RNA that is complementary to a region of target nucleic acid. Therapeutical use of intracellularly expressed antisense RNA is procedurally similar to gene therapy.

In further preferred embodiments, the method comprises grafting donor cells into the central nervous system, preferably the brain, of said subject, or donor cells preferably treated so as to minimize or reduce graft rejection, wherein said donor cells are genetically modified by insertion of at least one transgene encoding said agent or agents. Said transgene might be carried by a viral vector, in particular a retroviral vector. The transgene can be inserted into the donor cells by a nonviral physical transfection of DNA encoding a transgene, in particular by microinjection. Insertion of the transgene can also be performed by electroporation, chemically mediated transfection, in particular calcium phosphate transfection, liposomal mediated transfection, etc.

In preferred embodiments, said agent is a therapeutic protein which can be administered to said subject, preferably a human, by a process comprising introducing subject cells into said subject, said subject cells having been treated in vitro to insert a DNA segment encoding said therapeutic protein, said subject cells expressing in vivo in said subject a therapeutically effective amount of said therapeutic protein. Said DNA segment can be inserted into said cells in vitro by a viral vector, in particular a retroviral vector.

In preferred embodiments, the subject can be a human, an experimental animal, e.g. a mouse or a rat, a domestic animal, or a non-human primate. The experimental animal can be an animal model for a neurodegenerative disorder, e.g. a transgenic mouse with an Alzheimer's-type neuropathology.

In a further aspect, the invention features a modulator of an activity, or a level, or both said activity and said level of at least one substance which is selected from the group consisting of (i) a gene coding for a caveolae-associated integral membrane protein, and/or (ii) a transcription product of a gene coding for a caveolae-associated integral membrane protein, and/or (iii) a translation product of a gene coding for a caveolae-associated integral membrane protein, and/or (iv) a fragment or derivative of (i) to (iii).

In an additional aspect, the invention features a pharmaceutical composition comprising said modulator and preferably a pharmaceutical carrier. Said carrier refers to a diluent, adjuvant, excipient, or vehicle with which the modulator is administered.

In a further aspect, the invention features a modulator of an activity, or a level, or both said activity and said level of at least one substance which is selected from the group consisting of (i) a gene coding for a caveolae-associated integral membrane protein, and/or (ii) a transcription product of a gene coding for a caveolae-associated integral membrane protein, and/or (iii) a translation product of a gene coding for a caveolae-associated integral membrane protein, and/or (iv) a fragment or derivative of (i) to (iii) for use in a pharmaceutical composition.

In another aspect, the invention provides for the use of a modulator of an activity, or a level, or both said activity and said level of at least one substance which is selected from the group consisting of (i) a gene coding for a caveolae-associated integral membrane protein, and/or (ii) a transcription product of a gene coding for a caveolae-associated integral membrane protein, and/or (iii) a translation product of a gene coding for a caveolae-associated integral membrane protein, and/or (iv) a fragment or derivative of (i) to (iii) for a preparation of a medicament for treating or preventing a neurodegenerative disease, in particular Alzheimer's disease.

In one aspect, the present invention also provides a kit comprising one or more containers filled with a therapeutically or prophylactically effective amount of said pharmaceutical composition.

In a further aspect, the invention features a recombinant, non-human animal comprising a non-native gene sequence coding for a caveolae-associated integral membrane protein, or a fragment thereof, or a derivative thereof. The generation of said recombinant, non-human animal comprises (i) providing a gene targeting construct containing said gene sequence and a selectable marker sequence, and (ii) introducing said targeting construct into a stem cell of a non-human animal, and (iii) introducing said non-human animal stem cell into a non-human embryo, and (iv) transplanting said embryo into a pseudo-pregnant non-human animal, and (v) allowing said embryo to develop to term, and (vi) identifying a genetically altered non-human animal whose genome comprises a modification of said gene sequence in both alleles, and (vii) breeding the genetically altered non-human animal of step (vi) to obtain a genetically altered non-human animal whose genome comprises a modification of said endogenous gene, wherein said gene is mis-expressed, or under-expressed, or over-expressed, and wherein said disruption or alteration results in said non-human animal exhibiting a predisposition to developing symptoms of neuropathology similar to a neurodegenerative disease, in particular Alzheimer's disease. Strategies and techniques for the generation and construction of such an animal are known to those of ordinary skill in the art (see e.g. Capecchi, Science, 1989, 244:1288-1292 and Hogan et al., 1994, Manipulating the Mouse Embryo: A Laboratory Manual, Cold Spring Harbor Laboratory Press, Cold Spring Harbor, N.Y.; the contents of the foregoing are incorporated herein by reference). It is preferred to make use of such a recombinant non-human animal as an animal model for investigating neurodegenerative diseases, in particular Alzheimer's disease.

In preferred embodiments, said recombinant, non-human animal comprises a non-native gene sequence coding for a member of the flotillin gene family, in particular the caveolae-associated integral membrane protein flotillin-1, or a fragment thereof, in particular FCRD.

In another aspect, the invention features an assay for screening for a modulator of neurodegenerative diseases, in particular Alzheimer's disease, or related diseases and-disorders of one or more substances selected from the group consisting of (i) a gene coding for a caveolae-associated integral membrane protein, and/or (ii) a transcription product of a gene coding for a caveolae-associated integral membrane protein, and/or (iii) a translation product of a gene coding for a caveolae-associated integral membrane protein, and/or (iv) a fragment or derivative of (i) to (iii). This screening method comprises (a) contacting a cell with a test compound, and (b) measuring the activity, or the level, or both the activity and the level of one or more substances recited in (i) to (iv), and (c) measuring the activity, or the level, or both the activity and the level of said substances in a control cell not contacted with said test compound, and (d) comparing the levels of the substance in the cells of step (b) and (c), wherein an alteration in the activity and/or level of said substances in the contacted cells indicates that the test compound is a modulator of said diseases and disorders.

In one further aspect, the invention features a screening assay for a modulator of neurodegenerative diseases, in particular Alzheimer's disease, or related diseases and disorders of one or more substances selected from the group consisting of (i) a gene coding for a caveolae-associated integral membrane protein, and/or (ii) a transcription product of a gene coding for a caveolae-associated integral membrane protein, and/or (iii) a translation product of a gene coding for a caveolae-associated integral membrane protein, and/or (iv) a fragment or derivative of (1) to (iii), comprising (a) administering a test compound to a test animal which is predisposed to developing or has already developed a neurodegenerative disease or related diseases or disorders, and (b) measuring the activity and/or level of one or more substances recited in (i) to (iv), and (c) measuring the activity and/or level of said substances in a matched control animal which is equally predisposed to developing or has already developed said diseases and to which animal no such test compound has been administered, and (d) comparing the activity and/or level of the substance in the animals of step (b) and (c), wherein an alteration in the activity and/or level of substances in the test animal indicates that the test compound is a modulator of said diseases and disorders.

In another embodiment, the present invention provides a method for producing a medicament comprising the steps of (i) identifying a modulator of neurodegenerative diseases by a method of the aforementioned screening assays and (ii) admixing the modulator with a pharmaceutical carrier. Said modulator may also be identifiable by other assays of screening.

In a preferred embodiment, said test animal and/or said control animal is a recombinant, non-human animal which expresses a caveolae-associated integral membrane protein, or a fragment thereof, or a derivative thereof, under the control of a transcriptional regulatory element which is not the native caveloae-associated integral membrane protein gene transcriptional control regulatory element.

In another aspect, the present invention provides for an assay for screening a plurality of compounds for inhibition of binding between a ligand and a caveolae-associated integral membrane protein, or a fragment or derivative thereof. Said screening assay comprises the steps of (1) adding a liquid suspension of said caveolae-associated integral membrane protein, or a fragment or derivative thereof, to a plurality of containers, and (ii) adding a plurality of compounds to be screened for said inhibition to said plurality of containers, and (iii) adding fluorescently detectable ligand to said containers, and (iv) incubating said caveolae-associated integral membrane protein, or said fragment or derivative thereof, and said compounds, and said fluorescently labelled ligand, and (v) measuring the amounts of fluorescence associated with said caveolae-associated integral membrane protein, or with said fragment or derivative thereof, and (vi) determining the degree of inhibition by one or more of said compounds of binding of said ligand to said caveolae-associated integral membrane protein, or said fragment or derivative thereof. Instead of utilizing a fluorescently labelled ligand, it might in some aspects be preferred to use any other detectable label known to the person skilled in the art, e.g. radioactive labels, and detect it accordingly.

In one further embodiment, the present invention provides a method for producing a medicament comprising the steps of (i) identifying a compound as an inhibitor by the aforementioned method of inhibitory binding assays and (ii) admixing the compound with a pharmaceutical carrier. Said compound may also be identifiable by other assays of screening.

In one further aspect, the invention features an assay for screening a plurality of compounds to determine the degree of binding of said compounds to a caveolae-associated integral membrane protein, or to a fragment or derivative thereof. Said screening assay comprises (i) adding a liquid suspension of said caveolae-associated integral membrane protein, or a fragment or derivative thereof, to a plurality of containers, and (ii) adding a plurality of fluorescently detectable compounds to be screened for said binding to said plurality of containers, and (iii) incubating said caveolae-associated integral membrane protein, or said fragment or derivative thereof, and said fluorescently labelled compounds, and (iv) measuring the amounts of fluorescence associated with said caveolae-associated integral membrane protein, or with said fragment or derivative thereof, and (v) determining the degree of binding by one or more of said compounds to said caveolae-associated integral membrane protein, or said fragment or derivative thereof. Also in this type of assay it might be preferred to use another type of label.

In one further embodiment, the present invention provides a method for producing a medicament comprising the steps of (i) identifying a compound as a binder to a caveolae-associated integral membrane protein by the aforementioned method of binding assays and (ii) admixing the compound with a pharmaceutical carrier. Said compound may also be identifiable by other assays of screening.

In another embodiment, the present invention provides for a medicament obtainable by any of the methods according to the herein claimed screening assays.

In one further embodiment, the instant invention provides a medicament obtained by any of the methods according to the herein claimed screening assays.

In all types of assays disclosed herein it is preferred to study a member of the flotillin gene family as said caveolae-associated integral membrane protein. It is particularly preferred to conduct screening assays with flotillin-1.

Other features and advantages of the invention will be apparent from the following description of figures and examples.

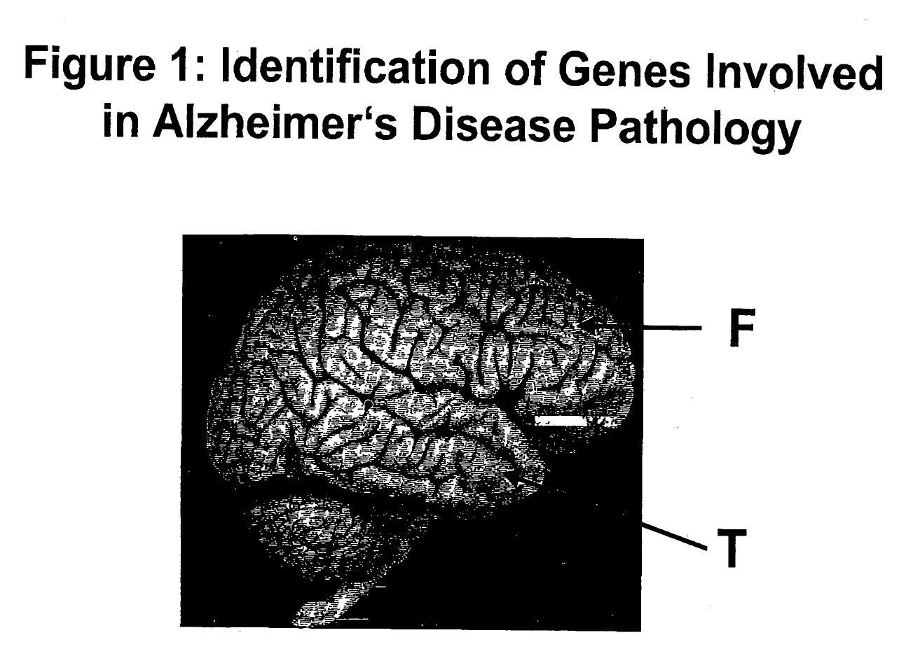

FIG. 1 depicts the brain regions with selective vulnerability to neuronal loss and degeneration in Alzheimer's disease. Primarily neurons within the inferior temporal lobe, the entorhinal cortex, the hippocampus, and the amygdala are subject to degenerative processes in Alzheimer's disease (Terry et al., Annals of Neurology 1981, 10:184-192). These brain regions are mostly involved in the processing of learning and memory functions. In contrast, neurons within the frontal cortex, the occipital cortex; and the cerebellum remain largely intact and preserved from neurodegenerative processes in Alzheimer's disease. Brain tissues from the frontal cortex (F) and the temporal cortex (T) of Alzheimer's disease patients and healthy, age-matched control individuals were used for the herein disclosed examples. For illustrative purposes, the image of a normal, healthy brain was taken from a publication by Strange PG (1992, Brain Biochem. Brain Disord.).

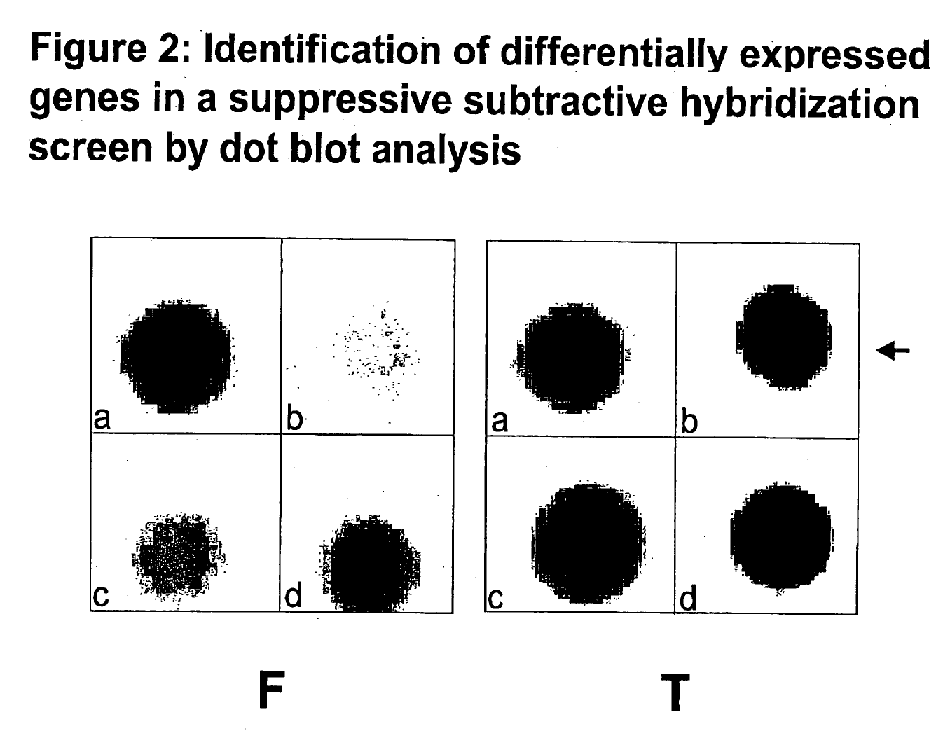

FIG. 2 discloses the initial identification of the differential expression of flotillin-1 in a suppressive subtractive hybridization screen. The figure shows a clipping of a large-scale dot blot hybridization experiment. Individual cDNA clones from a temporally subtracted library were arrayed onto a nylon membrane and hybridized with probes enriched for genes expressed in the frontal cortex (F) and the temporal cortex (T) from an Alzheimer's disease patient. a) clone T10-D06; b) flotillin-1; c) clone ens-0144; d) clone T10-E07. Note the significant increase in intensity of the hybridization signal for flotillin-1 in panel (T) (see arrow head) as compared to the signal in panel (F). Note that knowledge of the identity of clones T10-D06, ens-0144, and T10-E07 is not critical for the technical feasibility of the experiment.

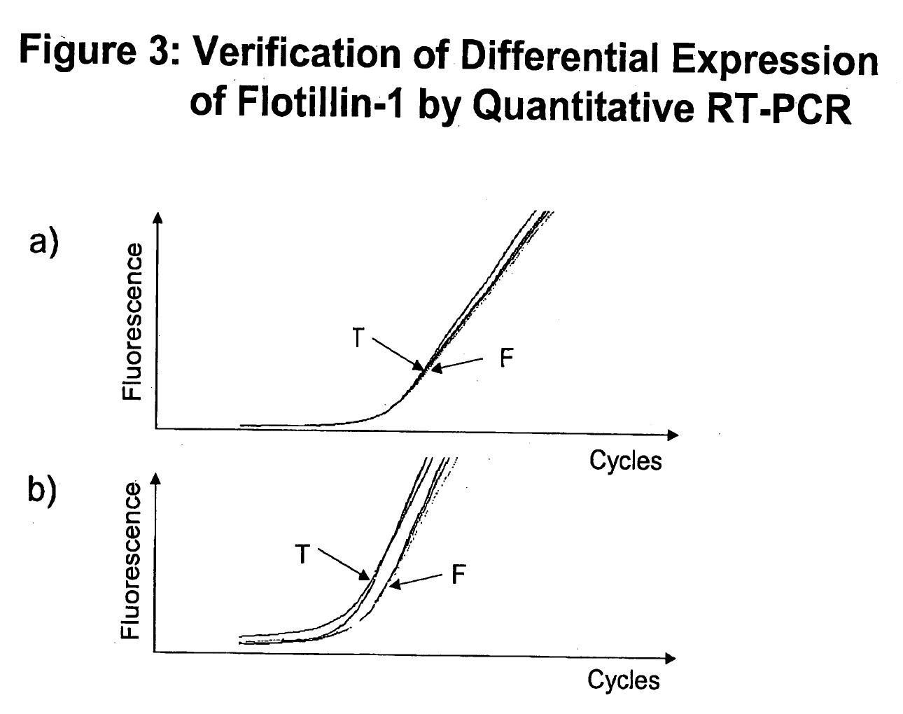

FIG. 3 illustrates the verification of the differential expression of flotillin-1 by quantitative RT-PCR analysis. Quantification of RT-PCR products from RNA samples collected from the frontal cortex (F) and temporal cortex (T) of healthy, age-matched control individuals (FIG. 3a) and Alzheimer's disease patients (FIG. 3b) was performed by the LightCycler™ rapid thermal cycling technique. The data were normalized to cyclophilin B which showed no significant difference in its gene expression level. The figure depicts the kinetics of amplification by plotting the cycle number against the amount of amplified material as measured by its fluorescence. Note that the amplification kinetics of flotillin-1 cDNA from both the frontal and temporal cortices of a normal control individual during the exponential phase of the reaction overlap, whereas in Alzheimer's disease (FIG. 3b) there is a significant shift of the curve for the sample derived from temporal cortex, indicating an up-regulation of flotillin-1 mRNA expression in temporal cortex in comparison to frontal cortex.

Table 1 lists the up-regulation of gene expression levels in the temporal cortex relative to the frontal cortex for the flotillin-1 gene in two Alzheimer's disease patients and two healthy, age-matched control individuals.

### EXAMPLE I

(i) Brain Tissue Dissection From Patients with Alzheimer's Disease:

Brain tissues from Alzheimer's disease patients and age-matched control subjects were collected within 6 hours of death and immediately frozen on dry ice. Sample sections from each tissue were fixed in paraformaldehyde for histopathological confirmation of the diagnosis. Brain areas for differential expression analysis were identified (see FIG. 1) and stored at −80° C. until RNA extractions were performed.

(ii) Isolation of Total mRNA:

Total RNA was extracted from post-mortem brain tissue by using the RNeasy™ kit (Qiagen) according to the manufacturer's protocol. The quality of the prepared RNA was determined by formaldehyde agarose gel electrophoresis and Northern blotting according to standard procedures (Sambrook and Russell, Molecular Cloning: A Laboratory Manual, Cold Spring Harbor Laboratory Press, Cold Spring Harbor, N.Y., 2000). The mRNA was isolated from the total RNA preparation using the Quickprep Micro™ mRNA Purification Kit (Pharmacia Biotech) with yields between 1 and 5%.

(iii) cDNA Synthesis and Identification of Differentially Expressed Genes by Suppressive Subtractive Hybridization:

This technique compares two populations of mRNA and provides clones of genes that are expressed in one population but not in the other. The applied technique was described in detail by Diatchenko et al. (Proc Natl Acad Sci USA 1996, 93:6025-30) In the present invention, mRNA populations from post-mortem brain tissues from Alzheimer's disease patients were compared. Specifically, mRNA of the frontal cortex was subtracted from mRNA of the inferior temporal cortex. The necessary reagents were taken from the PCR-Select™ cDNA subtraction kit (Clontech), and all steps were performed as described in the manufacturer's protocol. Specifically, 2μg mRNA each were used for first-strand and second-strand cDNA synthesis. After RsaI-digestion and adaptor ligation hybridization of tester and driver was performed for 8 hours (first hybridization) and 15 hours (second hybridization) at 68° C. Two PCR steps were performed to amplify differentially expressed genes (first PCR: 27 cycles of 94° C. and 30 sec, 66° C. and 30 sec, and 72° C. and 1.5 min; nested PCR: 12 cycles of 94° C. and 30 sec, 66° C. and 30 sec, and 72° C. and 1.5 min) using adaptor specific primers (included in the subtraction kit) and 50× Advantage Polymerase Mix (Clontech). Efficiencies of RsaI-digestions, adaptor ligations and subtractive hybridizations were checked as recommended in the kit. Subtracted cDNAs were inserted into the pCR® vector and transformed into E. coli INVaF′ cells (Invitrogen).

To isolate individual cDNAs of the subtracted library single bacterial transformants were incubated in 100 μl LB (with 50 μg/ml ampicillin) at 37° C. for at least 4 hours. Inserts were PCR amplified (95° C. and 30 sec, 68° C. and 3 min for 30 cycles) in a volume of 20 μl containing 10 mM Tris-HCl pH 9.0, 1.5 mM MgCl₂, 50 mM KCl, 200 μM dNTP, 0.5 μM adaptor specific primers (included in the subtraction kit), 1.5 Units Taq polymerase (Pharmacia Biotech), and 1 μl of bacterial culture.

1.5 μl of a mixture containing 3 μl PCR amplified inserts and 2 μl 0.3 N NaOH/15% Ficoll were spotted onto a positively charged nylon membrane (Roche). In this way, hundreds of spots were arrayed on duplicate filters for subsequent hybridization analysis. The differential screening step consisted of hybridizations of the subtracted library with itself to minimize background (Wang and Brown, Proc Natl Acad Sci USA 1991, 88:11505-9). The probes were generated from the nested PCR product of the subtraction following the instructions of the Clontech subtraction kit. Labeling with digoxigenin was performed with the DIG DNA Labeling Kit (Roche). Hybridizations were carried out overnight in DIG Easy HYB (Roche) at 43° C. The filters were washed twice in 2×SSC/0.5 % SDS at 68° C. for 15 min and twice in 0.1×SSC/0.5% SDS at 68° C. for 15 min, and subjected to detection using anti-DIG-AP conjugates and CDP-Star™ as chemiluminescent substrate according to the instructions of the DIG DNA Detection Kit (Roche). Blots were exposed to Kodak Blomax MR chemiluminescent film at room temperature for several minutes. The nucleotide sequences of clones of interest were obtained using methods well known to those skilled in the art. For nucleotide sequence analyses and homology searches, computer algorithms of the University of Wisconsin Genetics Computer Group (GCG) in conjunction with publicly available nucleotide and peptide sequence information (Genbank and EMBL databases) were employed. The results of one such subtractive hybridization experiment for the flotillin-1 gene are shown in FIG. 2.

(iv) Confirmation of Differential Expression by Quantitative RT-PCR:

Positive corroboration of differential expression of the flotillin-1 gene was performed using the LightCycler™ technology (Roche). This technique features rapid thermal cyling for the polymerase chain reaction as well as real-time measurement of fluorescent signals during amplification and therefore allows for highly accurate quantification of RT-PCR products by using a kinetic, rather than an endpoint approach. The ratio of flotillin-1 cDNA from the temporal cortex and frontal cortex was determined (relative quantification).

First, a standard curve was generated to determine the efficiency of the PCR with specific primers for flotillin-1 (5′-CTGCCAGAGAGTGTGGAAAGACT-3′ and 5′-TCTCAAAGGCTTGTGATTCACCT-3′). PCR amplification (95° C. and 1 sec, 56° C. and 5 sec, and 72° C. and 5 sec) was performed in a volume of 20 μl containing Lightcycler-DNA Master SYBR Green ready-to-use mix (contains Taq DNA polymerase, reaction buffer, dNTP mix with dUTP instead of dTTP, SYBR Green I dye, and 1 mM MgCl₂, Roche), additional 3 mM MgCl₂, 0.5 μM primers, 0.16 μl TaqStart® antibody (Clontech), and 1 μl of a cDNA dilution series (40, 20, 10, 5, and 1 ng human total brain cDNA, Clontech). Melting curve analysis revealed a single peak at approximately 83° C. with no visible primer dimers. Agarose gel analysis of the PCR product showed one single band of the expected size (66 bp).

The same protocol was applied to determine the PCR efficiency of the reference gene, cyclophilin B, using the specific primers 5′-ACTGAAGCACTACGGGCCTG-3′ and 5′-AGCCGTTGGTGTCTTTGCC-3′ except for MgCl₂ (additional 1 mM was added instead of 3 mM). Cyclophilin-B was chosen for normalization because it was found to be the least regulated gene among all analyzed housekeeping genes. Melting curve analysis revealed a single peak at approximately 87° C. with no visible primer dimers. Agarose gel analysis of the PCR product showed one single band of the expected size (62 bp).

The logarithm of the cDNA concentration was plotted against the threshold cycle number Cₜ for both flotillin-1 and cyclophilin B. The slopes and the intercepts of the standard curves (linear regressions) were calculated for both genes.

In a second step, cDNA from temporal cortex and frontal cortex was analyzed in parallel with cyclophilin B for normalization. The Cₜ values were measured and converted to ng total brain cDNA using the corresponding standard curves:

10\hat{ }((Cₜ value−intercept)/slope) [ng total brain cDNA]

The values of temporal and frontal cortex flotillin-1 cDNAs were normalized to cyclophilin B and the ratio was calculated using the following formula:

$$Ratio = \frac{flotillin \text{-} 1 temporal [ ng ] / cyclophilin B temporal [ ng ]}{flotillin \text{-} 1 frontal [ ng ] / cyclophilin B frontal [ ng ]} .$$

The results of one such quantitative RT-PCR analysis for the flotillin-1 gene are shown in FIG. 3.

1. A method of diagnosing or prognosing a neurodegenerative disease in a subject, or determining whether a subject is at increased risk of developing said disease, comprising:
determining a level and/or an activity of
(i) a transcription product of a gene coding for a caveolae-associated integral membrane protein, and/or
(ii) a translation product of a gene coding for a caveolae-associated integral membrane protein, and/or
(iii) a fragment or derivative of said transcription or translation product,
in a sample from said subject and comparing said level and/or said activity to a reference value representing a known disease or health status, thereby diagnosing or prognosing said neurodegenerative disease in said subject, or determining whether said subject is at increased risk of developing said neurodegenerative disease.

2. A method of monitoring the progression of a neurodegenerative disease in a subject, comprising;
determining a level and/or an activity of
(i) a transcription product of a gene coding for a caveolae-associated integral membrane protein, and/or
(ii) a translation product of a gene coding for a caveolae-associated integral membrane protein, and/or
(iii) a fragment or derivative of said transcription or translation product,
in a sample from said subject and comparing said level and/or said activity to a reference value representing a known disease or health status, thereby monitoring the progression of said neurodegenerative disease in said subject.

3. A method of evaluating a treatment for a neurodegenerative disease, comprising:
determining a level and/or an activity of
(i) a transcription product of a gene coding for a caveolae-associated integral membrane protein, and/or
(ii) a translation product of a gene coding for a caveolae-associated integral membrane protein, and/or
(iii) a fragment or derivative of said transcription or translation product,
in a sample from a subject being treated for said disease and comparing said level and/or said activity to a reference value representing a known disease or health status, thereby evaluating said treatment for said neurodegenerative disease.

4. The method according to any of claims 1 to 3 wherein said neurodegenerative disease is Alzheimer's disease.

5. The method according to any of claims 1 to 4 wherein said caveolae-associated integral membrane protein is a member of the flotillin gene family, in particular flotillin-1.

6. The method according to any of claims 1 to 5 wherein said fragment of said caveolae-associated integral membrane protein is a fragment of flotillin-1, in particular FCRD.

7. The method according to any of claims 1 to 6 wherein said sample is a cell, or a tissue, or a body fluid, in particular cerebrospinal fluid.

8. The method according to any of claims 1 to 7 wherein said reference value is that of a level and/or an activity of
(i) a transcription product of a gene coding for a caveolae-associated integral membrane protein, and/or
(ii) a translation product of a gene coding for a caveolae-associated integral membrane protein, and/or
(iii) a fragment or derivative of said transcription or translation product,
in a sample from a subject not suffering from said neurodegenerative disease.

9. The method according to any of claims 1 to 8 wherein an altered amount in flotillin-1 mRNA and/or flotillin-1 protein in a cell, or tissue, or body fluid from said subject relative to a reference value representing a known health status indicates a diagnosis, or prognosis, or increased risk of Alzheimer's disease in said subject.

10. The method according to any of claims 1 to 9 wherein said transcription product and/or a fragment or derivative of said transcription product is determined using a PCR-analysis and/or a Northern analysis.

11. The method according to any of claims 1 to 9 wherein said translation product and/or a fragment or derivative of said translation product is determined using an immunoassay, a Western blot analysis, an enzyme activity assay, and/or a binding assay.

12. The method according to any of claims 1 to 11, further comprising
comparing a level and/or an activity of
(i) a transcription product of a gene coding for a caveolae-associated integral membrane protein, and/or
(ii) a translation product of a gene coding for a caveolae-associated integral membrane protein, and/or
(iii) a fragment or derivative of said transcription or translation product
in a series of samples taken from said subject over a period of time.

13. The method according to claim 12 wherein said subject receives a treatment prior to one or more of said sample gatherings.

14. The method according to claim 13 wherein said level and/or activity is determined before and after said treatment of said subject.

15. A method of treating or preventing a neurodegenerative disease, in particular Alzheimer's disease, in a subject comprising administering to said subject in a therapeutically or prophylactically effective amount an agent or agents which directly or indirectly affect an activity and/or a level of (i) a gene coding for a caveolae-associated integral membrane protein, and/or (ii) a transcription product of a gene coding for a caveolae-associated integral membrane protein, and/or (iii) a translation product of a gene coding for a caveolae-associated integral membrane protein, and/or (iv) a fragment or derivative of (i) to (iii).

16. A modulator of an activity and/or of a level of at least one substance which is selected from the group consisting of (i) a gene coding for a caveolae-associated integral membrane protein, and/or (ii) a transcription product of a gene coding for a caveolae-associated integral membrane protein, and/or (iii) a translation product of a gene coding for a caveolae-associated integral membrane protein, and/or (iv) a fragment or derivative of (i) to (iii).

17. A pharmaceutical composition comprising a modulator according to claim 16.

18. A modulator of an activity and/or of a level of at least one substance which is selected from the group consisting of (i) a gene coding for a caveolae-associated integral membrane protein, and/or (ii) a transcription product of a gene coding for a caveolae-associated integral membrane protein, and/or (iii) a translation product of a gene coding for a caveolae-associated integral membrane protein, and/or (iv) a fragment or derivative of (i) to (iii) for use in a pharmaceutical composition.

19. Use of a modulator of an activity and/or of a level of at least one substance which is selected from the group consisting of (i) a gene coding for a caveolae-associated integral membrane protein, and/or (ii) a transcription product of a gene coding for a caveolae-associated integral membrane protein, and/or (iii) a translation product of a gene coding for a caveolae-associated integral membrane protein, and/or (iv) a fragment or derivative of (i) to (iii) for a preparation of a medicament for treating or preventing a neurodegenerative disease, in particular Alzheimer's disease.

20. A kit, comprising in one or more containers, a therapeutically or prophylactically effective amount of the pharmaceutical composition of claim 17.

21. A recombinant, non-human animal comprising a non-native gene sequence coding for a caveolae-associated integral membrane protein, or a fragment thereof, or a derivative thereof, said animal being obtainable by:
(i) providing a gene targeting construct comprising said gene sequence and a selectable marker sequence, and
(ii) introducing said targeting construct into a stem cell of a non-human animal, and
(iii) introducing said non-human animal stem cell into a non-human embryo, and
(iv) transplanting said embryo into a pseudopregnant non-human animal, and
(v) allowing said embryo to develop to term, and
(vi) identifying a genetically altered non-human animal whose genome comprises a modification of said gene sequence in both alleles, and
(vii) breeding the genetically altered mouse of step (vi) to obtain a genetically altered non-human animal whose genome comprises a modification of said endogenous gene, wherein said disruption results in said non-human animal exhibiting a predisposition to developing a neurodegenerative disease or related disease or disorders.

22. The animal according to claim 21 wherein said caveolae-associated integral membrane protein is a member of the flotillin gene family, in particular flotillin-1.

23. The animal according to claim 21 and 22 wherein said fragment of said caveolae-associated integral membrane protein is a fragment of flotillin-1, in particular FCRD.

24. An assay for screening for a modulator of neurodegenerative diseases, in particular Alzheimer's disease, or related diseases or disorders of one or more substances selected from the group consisting of
(i) a gene coding for a caveolae-associated integral membrane protein, and/or
(ii) a transcription product of a gene coding for a caveolae-associated integral membrane protein, and/or
(iii) a translation product of a gene coding for a caveolae-associated integral membrane protein, and/or
(iv) a fragment or derivative of (i) to (iii), said method comprising:
(a) contacting a cell with a test compound;
(b) measuring the activity and/or level of one or more substances recited in (i) to (iv);
(c) measuring the activity and/or level of one or more substances recited in (i) to (iv) in a control cell not contacted with said test compound; and
(d) comparing the levels and/or activities of the substance in the cells of step (b) and (c), wherein an alteration in the activity and/or level of substances in the contacted cells indicates that the test compound is a modulator of said diseases or disorders.

25. A method of screening for a modulator of neurodegenerative diseases, in particular Alzheimer's disease, or related diseases or disorders of one or more substances selected from the group consisting of
(i) a gene coding for a caveolae-associated integral membrane protein, and/or
(ii) a transcription product of a gene coding for a caveolae-associated integral membrane protein, and/or
(iii) a translation product of a gene coding for a caveolae-associated integral membrane protein, and/or
(iv) a fragment or derivative of (i) to (iii), said method comprising:
(a) administering a test compound to a test animal which is predisposed to developing or has already developed a neurodegenerative disease or related diseases or disorders in respect of the substances recited in (i) to (iv);
(b) measuring the activity and/or level of one or more substances recited in (i) to (iv);
(c) measuring the activity and/or level of one or more substances recited in (i) or (iv) in a matched control animal which is predisposed to developing or has already developed a neurodegenerative disease or related diseases or disorders in respect to the substances recited in (i) to (iv) and to which animal no such test compound has been administered;
(d) comparing the activity and/or level of the substance in the animals of step (b) and (c), wherein an alteration in the activity and/or level of substances in the test animal indicates that the test compound is a modulator of said diseases or disorders.

26. The method according to claim 25 wherein said test animal and/or said control animal is a recombinant animal which expresses a caveolae-associated integral membrane protein, or a fragment thereof, or a derivative thereof, under the control of a transcriptional control element which is not the native caveolae-associated integral membrane protein gene transcriptional control element.

27. An assay for screening a plurality of compounds for inhibition between a ligand and a caveolae-associated integral membrane protein, or a fragment or derivative thereof, said assay comprising the steps of:
(i) adding a liquid suspension of said caveolae-associated integral membrane protein, or a fragment or derivative thereof, to a plurality of containers;
(ii) adding a plurality of compounds to be screened for said inhibition to said plurality of containers;
(iii) adding a detectable ligand, in particular fluorescently labelled ligand, to said containers;
(iv) incubating said caveolae-associated integral membrane protein, or said fragment or derivative thereof, and said compounds, and said ligand;
(v) measuring amounts of detectable ligand or fluorescence associated with said caveolae-associated integral membrane protein, or with said fragment or derivative thereof; and
(vi) determining the degree of inhibition by one or more of said compounds of binding of said ligand to said caveolae-associated integral membrane protein, or said fragment or derivative thereof.

28. An assay for screening a plurality of compounds to determine the degree of binding of said compounds to a caveolae-associated integral membrane protein, or to a fragment or derivative thereof, said assay comprising the steps of:
(i) adding a liquid suspension of said caveolae-associated integral membrane protein, or a fragment or derivative thereof, to a plurality of containers;
(ii) adding a plurality of detectable compounds, in particular fluorescently labelled compounds, to be screened for said binding to said plurality of containers;
(iii) incubating said caveolae-associated integral membrane protein, or said fragment or derivative thereof, and said compounds;
(iv) measuring amounts of detectable compounds or fluorescence associated with said caveolae-associated integral membrane protein, or with said fragment or derivative thereof; and
(v) determining the degree of binding by one or more of said compounds to said caveolae-associated integral membrane protein, or said fragment or derivative thereof.

29. Use of the recombinant, non-human animal according to any of claims 21 to 23 as an animal model for investigating neurodegenerative diseases, in particular Alzheimer's disease.

30. A method for producing a medicament comprising the steps of (i) identifying a modulator of neurodegenerative diseases by a method according to claim 24 or 25 and (ii) admixing the modulator with a pharmaceutical carrier.

31. A method for producing a medicament comprising the steps of (i) identifying a compound as an inhibitor by a method according to claim 27 and (ii) admixing the compound with a pharmaceutical carrier.

32. A method for producing a medicament comprising the steps of (i) identifying a compound as a binder to a caveolae-associated integral membrane protein by a method according to claim 28 and (ii) admixing the compound with a pharmaceutical carrier.

33. A medicament obtainable by any of the methods according to claim 30 to 32.

34. A medicament obtained by any of the methods according to claim 30 to 32.
